(19) United States
(12) Patent Application Publication
(10) Pub. No.: US 2019/0002931 A1
(43) Pub. Date: Jan. 3, 2019

(54) METHOD FOR PRODUCING COMPOUND DERIVED FROM HERBACEOUS PLANT OF FAMILY GRAMINEAE OR CUCURBITACEAE

(71) Applicants: NITTO DENKO CORPORATION, Osaka (JP); NATIONAL UNIVERSITY CORPORATION KOBE UNIVERSITY, Hyogo (JP)

(72) Inventors: Kengo SASAKI, Hyogo (JP); Chiaki OGINO, Hyogo (JP); Akihiko KONDO, Hyogo (JP); Toshiyuki KAWASHIMA, Osaka (JP); Masahiko HIROSE, Osaka (JP); Takahisa KONISHI, Osaka (JP)

(73) Assignees: NITTO DENKO CORPORATION, Osaka (JP); NATIONAL UNIVERSITY CORPORATION KOBE UNIVERSITY, Hyogo (JP)

(21) Appl. No.: 15/752,780

(22) PCT Filed: Aug. 10, 2016
(86) PCT No.: PCT/JP2016/003714

§ 371 Date: Feb. 14, 2018

(30) Foreign Application Priority Data
Aug. 18, 2015 (JP) 2015-161388

(51) Int. Cl. C12P 7/14

(52) U.S. Cl. CPC C12P 7/14

(57) ABSTRACT

The production method of the present invention includes: a first fermentation step of adding at least one microorganism selected from yeast, Escherichia coli, and bacteria of the genus Corynebacterium to a first concentrated sugar solution to ferment the first concentrated sugar solution and subjecting the resulting first fermented solution to solid-liquid separation, the first concentrated sugar solution being obtained by using a nanofiltration membrane and/or a reverse osmosis membrane to concentrate a sugar solution obtained using a herbaceous plant of the family Gramineae or Cucurbitaceae as a raw material; and a second fermentation step of adding a second concentrated sugar solution to a solid component obtained in the first fermentation step to ferment the second concentrated sugar solution with the microorganism used in the first fermentation step, and subjecting the resulting second fermented solution to solid-liquid separation.

### TECHNICAL FIELD

The present invention relates to a method for producing a compound derived from a herbaceous plant of the family Gramineae or Cucurbitaceae.

### BACKGROUND ART

Herbaceous plants of the family Gramineae such as sorghum and herbaceous plants of the family Cucurbitaceae such as watermelon contain a high concentration of sugars. For example, sorghum can be grown in a short period and can be cultivated in a wide range of geographic areas due to its high tolerance to drought and salt, and thus the yield of sorghum is high. In recent years, therefore, bioethanol production by fermentation from juice of a herbaceous plant of the family Gramineae or Cucurbitaceae has attracted attention, and various techniques for such bioethanol production have been proposed.

For example, Non Patent Literature 1 proposes adding a sugar (sucrose) to sorghum juice and increasing the initial concentration of the sugar solution used for fermentation in order to produce a high concentration of ethanol from the sorghum juice and thereby reduce the amount of energy consumed in distillation and dehydration in the ethanol production process. Non Patent Literature 2 proposes concentrating sorghum juice through a two-stage membrane separation process and thereby increasing the initial concentration of the sugar solution used for fermentation.

Non Patent Literature 3 proposes a method for producing ethanol from sorghum juice by repeated-batch fermentation using yeast immobilized on corncob. In the method proposed in Non Patent Literature 3, each repetition of batch fermentation is performed using fresh sorghum juice supplemented with a nutrient source necessary for fermentation, as exemplified by a yeast extract, a nitrogen source such as peptone, and a sugar (sucrose).

### CITATION LIST

### Non Patent Literature

Non Patent Literature 1: L. Laopaiboon et al., “Ethanol production from sweet sorghum juice using very high gravity technology: Effects of carbon and nitrogen supplementations”, Bioresource Technology 100 (2009), 4176-4182

Non Patent Literature 2: K. Sasaki et al., “Increased ethanol production from sweet sorghum juice concentrated by a membrane separation process”, Bioresource Technology 169 (2014), 821-825

Non Patent Literature 3: L. Laopaiboon et al., “Ethanol production from sweet sorghum juice in repeated-batch fermentation by Saccharomyces cerevisiae immobilized on corncob”, World J Microbiol Biotechnol (2012) 28, 559-566

### SUMMARY OF INVENTION

### Technical Problem

As described above, various methods for producing ethanol from juice of a herbaceous plant such as sorghum by fermentation have conventionally been proposed. However, the conventional methods for producing a high concentration of ethanol from juice of a herbaceous plant such as sorghum leave room for improvement in terms of producing a high concentration of ethanol efficiently in a simple manner.

It is therefore an object of the present invention to provide a method with which a compound derived from a herbaceous plant of the family Gramineae or Cucurbitaceae, such as ethanol, can be produced efficiently and simply by fermentation using as a fermentation feedstock a sugar solution obtained using a herbaceous plant of the family Gramineae or Cucurbitaceae as a raw material.

### Solution to Problem

A first aspect of the present invention provides a method for producing a compound derived from a herbaceous plant of the family Gramineae or Cucurbitaceae, including:

a first fermentation step of adding at least one microorganism selected from yeast, Escherichia coli, and bacteria of the genus Corynebacterium to a first concentrated sugar solution to ferment the first concentrated sugar solution and subjecting the resulting first fermented solution to solid-liquid separation to separate the first fermented solution into a solid component and a liquid component, the first concentrated sugar solution being obtained by using at least one of a nanofiltration membrane and a reverse osmosis membrane to concentrate a sugar solution obtained using a herbaceous plant of the family Gramineae or Cucurbitaceae as a raw material; and

a second fermentation step of adding a second concentrated sugar solution to the solid component obtained in the first fermentation step to ferment the second concentrated sugar solution with the microorganism used in the first fermentation step, and subjecting the resulting second fermented solution to solid-liquid separation to separate the second fermented solution into a solid component and a liquid component, the second concentrated sugar solution being obtained by using at least one of a nanofiltration membrane and a reverse osmosis membrane to concentrate a sugar solution obtained using a herbaceous plant of the family Gramineae or Cucurbitaceae as a raw material,

wherein a compound derived from a herbaceous plant of the family Gramineae or Cucurbitaceae is obtained from the liquid component obtained in at least one fermentation step selected from the first fermentation step and the second fermentation step.

A second aspect of the present invention provides a method for producing a compound derived from a herbaceous plant of the family Gramineae or Cucurbitaceae, including:

a first fermentation step of adding at least one microorganism selected from yeast, Escherichia coli, and bacteria of the genus Corynebacterium to a first concentrated sugar solution to ferment the first concentrated sugar solution and subjecting the resulting first fermented solution to solid-liquid separation to separate the first fermented solution into a solid component and a liquid component, the first concentrated sugar solution being obtained by using at least one of a nanofiltration membrane and a reverse osmosis membrane to concentrate a sugar solution obtained using a herbaceous plant of the family Gramineae or Cucurbitaceae as a raw material;

a second fermentation step of adding a second concentrated sugar solution to the solid component obtained in the first fermentation step to ferment the second concentrated sugar solution with the microorganism used in the first fermentation step, and subjecting the resulting second fermented solution to solid-liquid separation to separate the second fermented solution into a solid component and a liquid component, the second concentrated sugar solution being obtained by using at least one of a nanofiltration membrane and a reverse osmosis membrane to concentrate a sugar solution obtained using a herbaceous plant of the family Gramineae or Cucurbitaceae as a raw material; and

third to N-th fermentation steps performed after the second fermentation step, where N is an integer of 3 or more,

wherein in an L-th fermentation step included in the second to N-th fermentation steps, where L is an integer satisfying 2≤L≤N, an L-th concentrated sugar solution obtained by using at least one of a nanofiltration membrane and a reverse osmosis membrane to concentrate a sugar solution obtained using a herbaceous plant of the family Gramineae or Cucurbitaceae as a raw material is added to a solid component obtained by solid-liquid separation of a fermented solution obtained in a fermentation step selected from the first to (L−1)-th fermentation steps to ferment the L-th concentrated sugar solution with the microorganism used in the fermentation step selected from the first to (L−1)-th fermentation steps, and the resulting L-th fermented solution is subjected to solid-liquid separation to separate the L-th fermented solution into a solid component and a liquid component, and

wherein a compound derived from a herbaceous plant of the family Gramineae or Cucurbitaceae is obtained from the liquid component obtained in at least one fermentation step selected from the first to N-th fermentation steps.

### Advantageous Effects of Invention

The production method according to the first aspect of the present invention is a method for producing a compound derived from a herbaceous plant of the family Gramineae or Cucurbitaceae by repeated-batch fermentation, the method including at least the first fermentation step and the second fermentation step. In the production method according to the first aspect of the present invention, the fermentation feedstock used for the first fermentation step and the second fermentation step is a concentrated sugar solution obtained by using a particular membrane to concentrate a sugar solution obtained using a herbaceous plant of the family Gramineae or Cucurbitaceae as a raw material. Thus, fermentation can be allowed to proceed to produce a compound derived from a herbaceous plant of the family Gramineae or Cucurbitaceae without supplementation with a nutrient source such as a nitrogen source or with an additional microorganism, despite the fact that the microorganism used in the first fermentation step is repeatedly used in the second fermentation step. The production method according to the second aspect of the present invention, in which three or more fermentation steps are performed, also employs as a fermentation feedstock a concentrated sugar solution obtained by using a particular membrane to concentrate a sugar solution obtained using a herbaceous plant of the family Gramineae or Cucurbitaceae as a raw material. Thus, as in the production method according to the first aspect of the present invention, a compound derived from a herbaceous plant of the family Gramineae or Cucurbitaceae can be produced by repeatedly using a microorganism without supplementation with a nutrient source such as a nitrogen source or with an additional microorganism. The production methods according to the first and second aspects of the present invention, which, as described above, permit repeated use of a microorganism for fermentation and eliminate the need for supplementation with a nutrient source such as a nitrogen source or with an additional microorganism, are capable of producing a compound derived from a herbaceous plant of the family Gramineae or Cucurbitaceae more efficiently and more simply than conventional methods.

### BRIEF DESCRIPTION OF DRAWINGS

FIG. 1 is a flow diagram showing the outline of a method for producing a sorghum-derived compound according to a first embodiment of the method of the present invention for producing a compound derived from a herbaceous plant of the family Gramineae or Cucurbitaceae.

FIG. 2 is a flow diagram showing the outline of a method for producing a sorghum-derived compound according to a second embodiment of the method of the present invention for producing a compound derived from a herbaceous plant of the family Gramineae or Cucurbitaceae.

FIG. 3 is a schematic diagram showing an apparatus used in membrane concentration performed in Examples and Comparative Examples.

FIG. 4A is a graph showing the concentrations of ethanol and glycerol in fermented solutions of Example 1.

FIG. 4B is a graph showing the concentrations of sucrose, glucose, and fructose in the fermented solutions of Example 1.

FIG. 5 is a graph showing the concentrations of sucrose, glucose, fructose, ethanol, and glycerol in fermented solutions of Example 2.

FIG. 6 is a graph showing a result of turbidimetric determination of yeast concentration in the fermented solutions of Example 2.

FIG. 7A is a graph showing the concentrations of ethanol and glycerol in fermented solutions of Comparative Example 1.

FIG. 7B is a graph showing the concentrations of sucrose, glucose, and fructose in the fermented solutions of Comparative Example 1.

FIG. 8A is a graph showing the concentrations of ethanol and glycerol in fermented solutions of Comparative Example 2.

FIG. 8B is a graph showing the concentrations of sucrose, glucose, and fructose in the fermented solutions of Comparative Example 2.

FIG. 9A is a graph showing the concentrations of ethanol and glycerol in fermented solutions of Example 3.

FIG. 9B is a graph showing the concentrations of sucrose, glucose, and fructose in the fermented solutions of Example 3.

### DESCRIPTION OF EMBODIMENTS

Hereinafter, embodiments of the present invention will be described. The following description is not intended to limit the present invention.

### First Embodiment

The method for producing a compound derived from a herbaceous plant of the family Gramineae or Cucurbitaceae according to the first embodiment includes:

a first fermentation step of adding at least one microorganism selected from yeast, Escherichia coli, and bacteria of the genus Corynebacterium to a first concentrated sugar solution to ferment the first concentrated sugar solution and subjecting the resulting first fermented solution to solid-liquid separation to separate the first fermented solution into a solid component and a liquid component, the first concentrated sugar solution being obtained by using at least one of a nanofiltration membrane (NF membrane) and a reverse osmosis membrane (RO membrane) to concentrate a sugar solution obtained using a herbaceous plant of the family Gramineae or Cucurbitaceae as a raw material; and

a second fermentation step of adding a second concentrated sugar solution to the solid component obtained by the solid-liquid separation of the first fermented solution obtained in the first fermentation step to ferment the second concentrated sugar solution with the microorganism used in the first fermentation step, and subjecting the resulting second fermented solution to solid-liquid separation to separate the second fermented solution into a solid component and a liquid component, the second concentrated sugar solution being obtained by using at least one of a NF membrane and a RO membrane to concentrate a sugar solution obtained using a herbaceous plant of the family Gramineae or Cucurbitaceae as a raw material,

wherein a compound derived from a herbaceous plant of the family Gramineae or Cucurbitaceae is obtained from a liquid component obtained in at least one fermentation step selected from the first fermentation step and the second fermentation step.

The production method of the present embodiment is a method for producing a compound derived from a herbaceous plant of the family Gramineae or Cucurbitaceae by repeated-batch fermentation, the method including at least the first fermentation step and the second fermentation step. The production method of the present embodiment employs as a fermentation feedstock a concentrated sugar solution obtained by using a particular membrane (at least one of a NF membrane and a RO membrane) to concentrate a sugar solution obtained using a herbaceous plant of the family Gramineae or Cucurbitaceae as a raw material. This concentrated sugar solution permits microbial culture without supplementation with a nutrient source, such as a nitrogen source, necessary for microbial culture. By virtue of the use of this concentrated sugar solution as a fermentation feedstock, the production method of the present embodiment allows fermentation to proceed to produce a compound derived from a herbaceous plant of the family Gramineae or Cucurbitaceae without supplementation with a nutrient source such as a nitrogen source, despite the fact that the microorganism used in the first fermentation step is repeatedly used in the second fermentation step. Additionally, the use of the concentrated sugar solution allows microbial culture to proceed along with fermentation, thus eliminating the need to add an additional microorganism in the second fermentation step. The reason why microbial culture is possible without supplementation of the concentrated sugar solution with a nitrogen source has not been clarified; however, it is presumed that as the sugar solution obtained using a herbaceous plant of the family Gramineae or Cucurbitaceae as a raw material is concentrated using the particular membrane mentioned above, not only sugars but also nutrient sources such as amino acids necessary for microbial culture are concentrated enough to allow the microbial culture to proceed without supplementation with an additional nitrogen source or the like.

Unlike the production method of the present embodiment, conventional methods for producing a plant-derived compound such as ethanol by repeated-batch fermentation use a common sugar solution obtained from sugar cane or sorghum juice as proposed in Non Patent Literature 3 without concentrating the sugar solution or sorghum juice with the particular membrane mentioned above. With such a sugar solution as used in the conventional production methods, it is difficult to accomplish microbial culture without supplementation with any nutrient source. The conventional production methods employing repeated-batch fermentation therefore require supplementation with a nutrient source such as a nitrogen source and supplementation with an additional microorganism for each repetition of fermentation.

As described above, the production method of the present embodiment permits repeated use of a microorganism for fermentation and does not require supplementation with a nutrient source such as nitrogen source or with an additional microorganism. The production method of the present embodiment is therefore capable of producing a compound derived from a herbaceous plant of the family Gramineae or Cucurbitaceae more efficiently and more simply than the conventional methods.

The production method of the present embodiment does not completely exclude the supplementation with an additional nutrient source or microorganism. A nutrient source or additional microorganism can be added to produce a compound derived from a herbaceous plant of the family Gramineae or Cucurbitaceae even more efficiently or even more simply.

In the method for producing a compound derived from a herbaceous plant of the family Gramineae or Cucurbitaceae according to the present embodiment, three or more fermentation steps may be performed. Namely, the method for producing a compound derived from a herbaceous plant of the family Gramineae or Cucurbitaceae according to the present embodiment may further include third to N-th fermentation steps performed after the second fermentation step, where N is an integer of 3 or more. In this case, in an L-th fermentation step included in the second to N-th fermentation steps, where L is an integer satisfying 2≤L≤N, an L-th concentrated sugar solution obtained by using at least one of a nanofiltration membrane and a reverse osmosis membrane to concentrate a sugar solution obtained using a herbaceous plant of the family Gramineae or Cucurbitaceae as a raw material is added to a solid component obtained by solid-liquid separation of a fermented solution obtained in a fermentation step selected from the first to (L−1)-th fermentation steps to ferment the L-th concentrated sugar solution with the microorganism used in the fermentation step selected from the first to (L−1)-th fermentation steps, and the resulting L-th fermented solution is subjected to solid-liquid separation to separate the L-th fermented solution into a solid component and a liquid component. When three or more fermentation steps are performed, it is recommended that a compound derived from a herbaceous plant of the family Gramineae or Cucurbitaceae be obtained from a liquid component obtained in at least one fermentation step selected from the first to N-th fermentation steps, rather than from a liquid component obtained in at least one fermentation step selected from the first fermentation step and the second fermentation step. When three or more fermentation steps are performed, the fermentation process in each fermentation step is desirably performed with the microorganism used in the immediately prior fermentation step in order to improve the efficiency and simplicity of the production method. That is, in the L-th fermentation step, it is desirable to add the L-th concentrated sugar solution to a solid component obtained in the (L−1)-th fermentation step and ferment the L-th concentrated sugar solution with the microorganism used in the (L−1)-th fermentation step.

An example of herbaceous plants of the family Gramineae is sorghum. Sorghum can be grown in a short period and can be cultivated in a wide range of geographic areas due to its high tolerance to drought and salt, and thus the yield of sorghum is high. Sorghum is therefore suitable as a biomass. The term “sorghum” as used herein encompasses a wide variety of annual grasses of the family Gramineae. When the production method of the present embodiment uses a sugar solution obtained using sorghum as a raw material, the sorghum used is suitably a sorghum such as white sorghum which has a high sugar content. An example of herbaceous plants of the family Cucurbitaceae is watermelon.

The term “compound derived from a herbaceous plant of the family Gramineae or Cucurbitaceae” as used herein encompasses all kinds of compounds which are obtained by employing as a fermentation feedstock a sugar solution produced using a herbaceous plant of the family Gramineae or Cucurbitaceae as a raw material and by fermenting the sugar solution. Examples of the compound derived from a herbaceous plant of the family Gramineae or Cucurbitaceae include: alcohols such as ethanol and butanol; lactic acid which is a bioplastic material; and succinic acid.

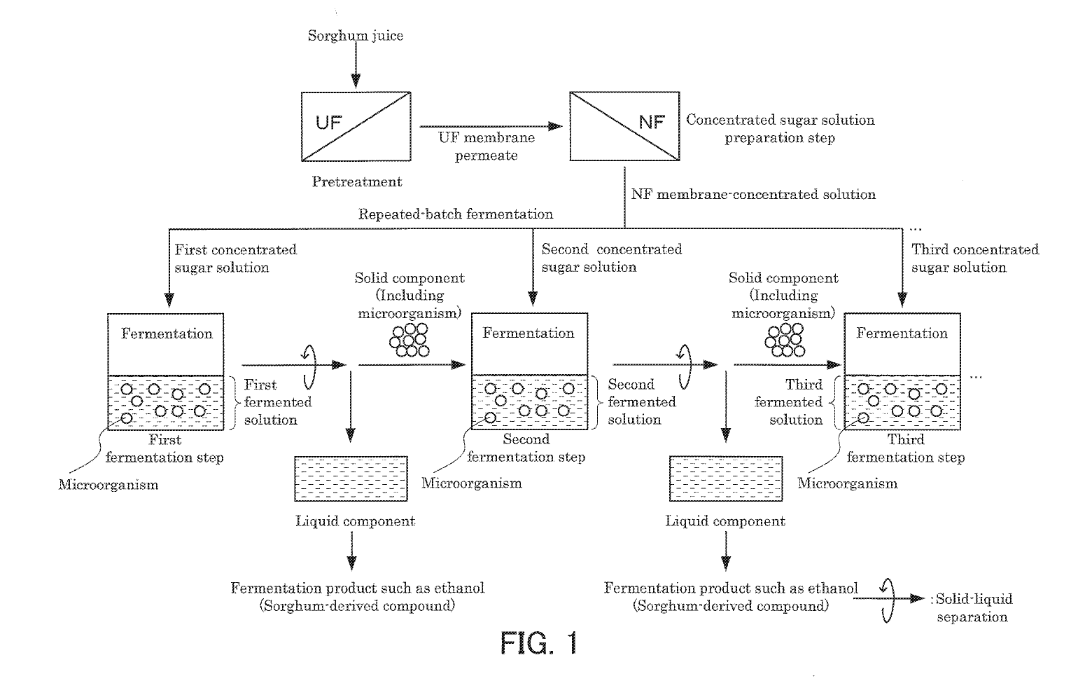

Hereinafter, an example of the production method of the present embodiment will be described with reference to the flow diagram of FIG. 1. The example described hereinafter is one in which sorghum is used as a raw material of a sugar solution and in which three or more repeated fermentation steps, namely the first to N-th fermentation steps, are performed. It should be noted that the production method of the present invention is not limited to the following example. Another herbaceous plant of the family Gramineae or Cucurbitaceae may be used instead of sorghum, or the number of repeated fermentation steps may be two.

First, a sugar solution obtained using sorghum as a raw material is prepared. An example of the sugar solution obtained using sorghum as a raw material is sorghum juice obtained by squeezing sorghum, and a known sugar solution made from sorghum can be used.

For example, sorghum juice obtained by squeezing sorghum often contains residues of the squeezed sorghum or dust. It is therefore desirable that the sugar solution obtained using sorghum as a raw material be subjected to a pretreatment in which the sugar solution is filtered through an ultrafiltration membrane (UF membrane) and the residue on the UF membrane is removed. The flow diagram of FIG. 1 shows an example in which the pretreatment is performed. The term “UF membrane” refers to a membrane with an average pore diameter of around 0.001 μm to 0.01 μm.

The material of the UF membrane is not particularly limited, and examples of usable materials include the following polymer materials: cellulose ester polymer such as cellulose acetate; polyethylene; polypropylene; polysulfone; polyvinylidene fluoride; and polyethersulfone. In view of durability and cleanability, polyvinylidene fluoride or polyethersulfone is preferred.

The form of the UF membrane is not particularly limited. A membrane selected from a flat membrane, a hollow fiber membrane, a pleated membrane, and a tubular membrane can be used. In particular, a so-called spiral membrane element, which is produced by forming a flat membrane into an envelope shape and spirally winding the envelope-shaped membrane together with a support such as a net, is preferred since the membrane element can provide a large membrane area.

The technique for filtering the sugar solution through the UF membrane is not particularly limited, and a known filtration technique using an UF membrane can be employed.

A concentrated sugar solution preparation step is subsequently performed. In the concentrated sugar solution preparation step, the sugar solution obtained using sorghum as a raw material is concentrated using at least one of a NF membrane and a RO membrane to prepare a concentrated sugar solution. The membrane concentration of the sugar solution can be accomplished by passing the sugar solution through at least one of a NF membrane and a RO membrane and by collecting the solution remaining on the feed side. The solution remaining on the feed side is a purified, concentrated solution of sugars, since substances having a lower molecular weight than sugars pass through the membrane.

The concentrated sugar solution prepared in the concentrated sugar solution preparation step can be used as the first to N-th concentrated sugar solutions in the first to N-th fermentation steps described later. The whole amount of the concentrated sugar solution to be used as the first to N-th concentrated sugar solutions may be prepared in the present step. Namely, as illustrated in the flow diagram of FIG. 1, a portion of the concentrated sugar solution obtained in the present step may be used as each of the first to N-th concentrated sugar solutions. Alternatively, a concentrated sugar solution may be prepared for each fermentation step.

The term “RO membrane” refers to a membrane that exhibits a sodium chloride removal efficiency of 93% or more in filtering a test solution with a sodium chloride concentration of 500 to 2,000 mg/L at an operation pressure of 0.5 to 3.0 MPa.

NF membranes generally have lower rejection performance than RO membranes and are characterized by excellent performance in removing divalent ions. NF membranes are semi-permeable membranes used also for filtration of organic substances or for decolorization. The term “NF membrane” refers to a membrane that exhibits a sodium chloride removal efficiency of 5% or more and less than 93% in filtering a test solution with a sodium chloride concentration of 500 to 2,000 mg/L at an operation pressure of 0.3 to 1.5 MPa.

The materials of the NF membrane and RO membrane are not particularly limited, and examples of usable materials include the following polymer materials: cellulose ester polymer such as cellulose acetate; polyamide; polyester; polyimide; vinyl polymer; polyethersulfone; sulfonated polyethersulfone; and polyamide. A plurality of materials may be used. Among the above polymer materials, for example, polyamide is suitably used for the RO membrane since the use of polyamide has been proved to provide high rejection performance. For the NF membrane, polyamide, polyethersulfone, polyvinyl alcohol, or a mixture thereof is suitably used.

The forms of the NF membrane and RO membrane are not particularly limited. Membranes selected from a flat membrane, a hollow fiber membrane, a pleated membrane, and a tubular membrane can be used. In particular, a so-called spiral membrane element, which is produced by forming a flat membrane into an envelope shape and spirally winding the envelope-shaped membrane together with a support such as a net, is preferred since the membrane element can provide a large membrane area.

The technique for membrane concentration using the NF membrane and/or RO membrane is not particularly limited, and a known membrane concentration technique can be employed. For example, membrane concentration may be successively repeated a plurality of times. In this case, the same type of membrane may be used in every repetition, or a different type of membrane may be used in each repetition. Performing the membrane concentration a plurality of times can result in an increase in the concentration of the concentrated sugar solution finally obtained. The membrane concentration using the NF membrane and/or RO membrane may thus be repeated a plurality of times until a desired sugar concentration is reached. The desired sugar concentration in the concentrated sugar solution finally obtained is, for example, 230 g/L or more.

Next, the first fermentation step is performed. In the first fermentation step, at least one microorganism selected from yeast, Escherichia coli, and bacteria of the genus Corynebacterium is added to a first concentrated sugar solution obtained by using at least one of a NF membrane and a RO membrane to concentrate a sugar solution obtained using sorghum as a raw material, and the first concentrated sugar solution is thus fermented. As previously described, a concentrated sugar solution prepared in the concentrated sugar solution preparation step can be used as the first concentrated sugar solution.

The fermentation using the first concentrated sugar solution as a fermentation feedstock can be accomplished using a known technique. The microorganism used for fermentation is at least one known microorganism selected from yeast, Escherichia coli, and bacteria of the genus Corynebacterium. The microorganism used may be identified from the natural environment or may be partially modified due to mutation or by genetic recombination. The microorganism used for fermentation may be preliminarily cultured up to an adequate concentration using a known culture medium such as YPD medium. The microorganism used for fermentation can be cultured using the concentrated sugar solution which is prepared in the present embodiment and which is produced from sorghum. For example, the production method of the present embodiment may include a culture step of culturing the microorganism for the first fermentation step, and the concentrated sugar solution as specified in the present embodiment (a concentrated sugar solution obtained by using at least one of a NF membrane and a RO membrane to concentrate a sugar solution obtained using a herbaceous plant of the family Gramineae or Cucurbitaceae as a raw material) can be used as the culture solution in the culture step. This culture step may be performed prior to the first fermentation step. The culture step may alternatively be performed simultaneously with fermentation in the first fermentation step; namely, the first fermentation step may include the culture step. The use of the concentrated sugar solution for microbial culture allows the microbial culture to proceed without supplementation with a nutrient source, such as a nitrogen source, necessary for the microbial culture. The reason why microbial culture is possible without supplementation of the concentrated sugar solution with a nitrogen source has not been clarified; however, it is presumed that as the sugar solution obtained using sorghum as a raw material is concentrated using a NF membrane and/or RO membrane, not only sugars but also nutrient sources such as amino acids necessary for microbial culture are concentrated enough to allow the microbial culture to proceed without supplementation with an additional nitrogen source or the like.

The first fermented solution obtained in the first fermentation step is subjected to solid-liquid separation to separate the first fermented solution into a solid component and a liquid component. The technique for the solid-liquid separation is not particularly limited, and a known technique for solid-liquid separation of fermented solutions, such as centrifugation, can be employed.

Next, the second fermentation step is performed. A second concentrated sugar solution obtained by using at least one of a NF membrane and a RO membrane to concentrate a sugar solution obtained using sorghum as a raw material is added to the solid component obtained in the first fermentation step, and the second concentrated sugar solution is thus fermented. The solid component obtained in the first fermentation step includes the microorganism. Thus, the microorganism used in the first fermentation step is used for fermentation of the second concentrated sugar solution. As previously described, a concentrated sugar solution prepared in the concentrated sugar solution preparation step can be used as the second concentrated sugar solution. The fermentation capacity of the microorganism is maintained without further adding any nitrogen source in conjunction with the addition of the second concentrated sugar solution to the solid component. In the second fermentation step, therefore, any additional nitrogen source need not be added to the solid component, and only the second concentrated sugar solution may be added for fermentation. In the second fermentation step, an additional microorganism may be added, although satisfactory fermentation can be achieved without supplementation with the additional microorganism.

The second fermented solution obtained in the second fermentation step is subjected to solid-liquid separation to separate the second fermented solution into a solid component and a liquid component. The technique for the solid-liquid separation is not particularly limited, and a known technique for solid-liquid separation of fermented solutions, such as centrifugation, can be employed.

Next, the third fermentation step is performed. A third concentrated sugar solution obtained by using at least one of a NF membrane and a RO membrane to concentrate a sugar solution obtained using sorghum as a raw material is added to the solid component obtained in the second fermentation step, and the third concentrated sugar solution is thus fermented. Namely, the microorganism used in the second fermentation step is used for fermentation of the third concentrated sugar solution. As previously described, a concentrated sugar solution prepared in the concentrated sugar solution preparation step can be used as the third concentrated sugar solution. The fermentation capacity of the microorganism is maintained without further adding any nitrogen source in conjunction with the addition of the third concentrated sugar solution to the solid component. In the third fermentation step, therefore, any additional nitrogen source need not be added to the solid component, and only the third concentrated sugar solution may be added for fermentation.

The third fermented solution obtained in the third fermentation step is subjected to solid-liquid separation to separate the third fermented solution into a solid component and a liquid component. The technique for the solid-liquid separation is not particularly limited, and a known technique for solid-liquid separation of fermented solutions, such as centrifugation, can be employed.

The fourth to N-th fermentation steps may subsequently be performed in the same manner as the third fermentation step. In the foregoing example, the fermentation process in a fermentation step is performed using a solid component (microorganism) obtained by solid-liquid separation of a fermented solution obtained in the immediately prior fermentation step. As previously described, the present invention is not necessarily limited to this example. Not only the microorganism used in the immediately prior fermentation step but also the microorganism used in the more prior fermentation step can be used.

A fermentation product such as ethanol (a compound derived from a herbaceous plant of the family Gramineae or Cucurbitaceae) is collected from the liquid component obtained by the solid-liquid separation in each fermentation step. The technique for collecting the fermentation product is not particularly limited, and a known collection technique can be appropriately selected depending on the fermentation product obtained.

As describe above, the method for producing a compound derived from a herbaceous plant of the family Gramineae or Cucurbitaceae according to the present embodiment performs repeated-batch fermentation using a concentrated sugar solution obtained by using a NF membrane and/or RO membrane to perform membrane concentration of a sugar solution obtained using a herbaceous plant of the family Gramineae or Cucurbitaceae as a raw material. This makes it possible to maintain the fermentation capacity of the microorganism and obtain a high concentration of fermentation product without supplementation with an additional nitrogen source.

### Second Embodiment

A method for producing a compound derived from a herbaceous plant of the family Gramineae or Cucurbitaceae according to the second embodiment includes the steps of the production method of the first embodiment and further includes, prior to the concentrated sugar solution preparation step, a sugar separation step of using a NF membrane to separate a sugar solution obtained using a herbaceous plant of the family Gramineae or Cucurbitaceae as a raw material into a sucrose-rich sugar solution and a reducing sugar-rich sugar solution. The reducing sugar-rich sugar solution obtained in this sugar separation step is used as the sugar solution in the concentrated sugar solution preparation step to prepare the concentrated sugar solution for use in the fermentation steps. The sucrose-rich sugar solution obtained in the sugar separation step can be used as a raw material for production of sugar. Thus, the production method of the present embodiment may include a sugar production step of producing sugar using the sucrose-rich sugar solution.

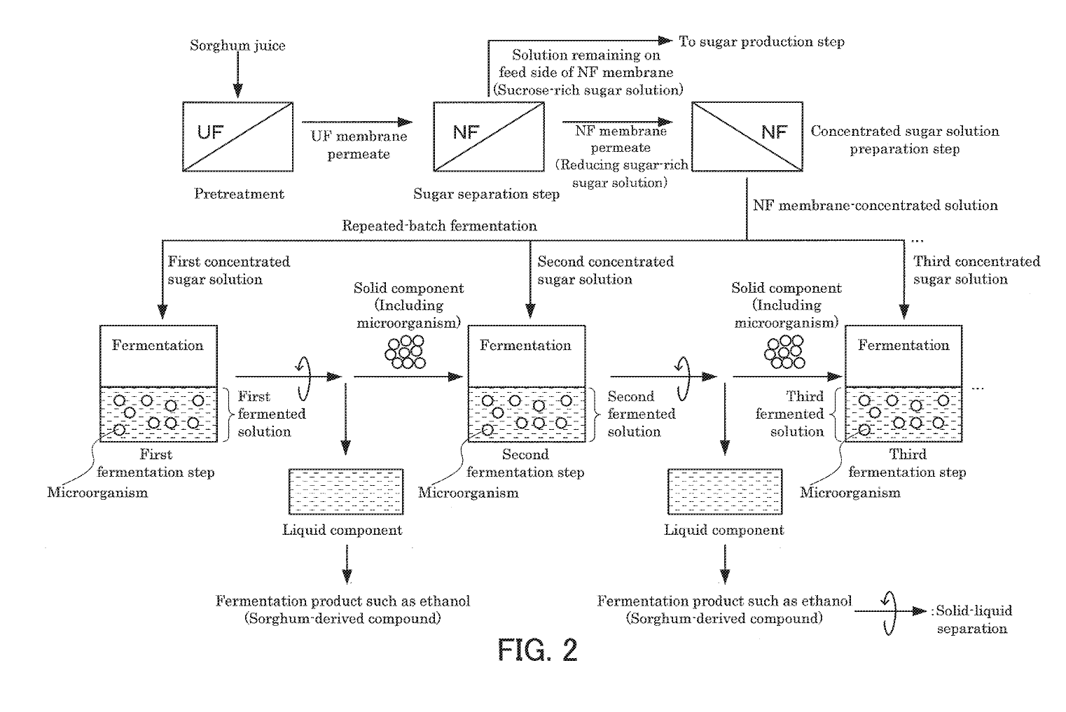

FIG. 2 is a flow diagram showing an example of the production method of the present embodiment. The production method shown as an example in FIG. 2 includes the steps of the production method shown as an example of the first embodiment in FIG. 1 and further includes the sugar separation step and sugar production step. The steps other than the sugar separation step and sugar production step are as described for the first embodiment, and only the sugar separation step and sugar production step will therefore be described hereinafter.

The sugar separation step is performed on a sugar solution that results from the UF membrane-assisted pretreatment of sorghum juice obtained by squeezing sorghum and that is not subjected to the concentrated sugar solution preparation step. In the sugar separation step, a NF membrane is used to separate the sugar solution into a sucrose-rich sugar solution and a reducing sugar-rich sugar solution. The NF membrane used in the sugar separation step is a membrane whose sucrose rejection performance is twice or more its glucose rejection performance. Namely, a NF membrane satisfying the following expression (1) is used in the sugar separation step.

Sucrose rejection performance/glucose rejection performance≥2 (1)

The term “sucrose rejection performance” as used to describe a NF membrane refers to a sucrose removal efficiency that the NF membrane exhibits in filtering a test solution with a sucrose concentration of 2000 ppm at an operation pressure of 1 MPa and a temperature of 25° C. The term “glucose rejection performance” refers to a glucose removal efficiency that the membrane exhibits in filtering a test solution with a glucose concentration of 2000 ppm at an operation pressure of 1 MPa and a temperature of 25° C.

The technique for the membrane-assisted sugar separation is not particularly limited, and a known membrane separation technique can be employed. For example, sugar separation may be successively repeated a plurality of times. In this case, the same type of membrane may be used in every repetition, or a different type of membrane may be used in each repetition. Performing the membrane-assisted sugar separation a plurality of times can lead to an increase in the degree of separation between sucrose and reducing sugars, thus resulting in an increase in the sucrose concentration of the sucrose-rich sugar solution and an increase in the reducing sugar concentration of the reducing sugar-rich sugar solution.

In the sugar separation step, reducing sugars can pass through the membrane, while sucrose is likely to remain on the feed side of the membrane by failing to pass through the membrane. Thus, the solution remaining on the feed side of the membrane can be collected as the sucrose-rich sugar solution. The solution passing through the membrane is collected as the reducing sugar-rich sugar solution and used as the sugar solution to be concentrated in the subsequent concentrated sugar solution preparation step. The concentrated sugar solution preparation step and the subsequent fermentation steps are as described for the first embodiment.

According to the flow diagram shown in FIG. 2, the sugar separation step is performed only once. However, the sugar separation step may be performed a plurality of times. For example, it is conceivable to collect the sugar solution remaining on the feed side of the membrane, dilute the collected sugar solution with water, pass the diluted sugar solution through a membrane for sugar separation, use the sugar solution having passed through the membrane as the reducing sugar-rich sugar solution in the sugar concentration step, and use the solution remaining on the feed side of the membrane as the sucrose-rich sugar solution in the sugar production step.

The sucrose-rich sugar solution obtained in the sugar separation step is used as a raw material for producing sugar in the sugar production step. The sugar production technique employed in the sugar production step can be a known sugar production technique, provided that the sucrose-rich sugar solution obtained from sorghum juice through the above step is used as a raw material.

With the production method of the present embodiment, a sugar solution obtained using a herbaceous plant of the family Gramineae or Cucurbitaceae as a raw material can be used not only as a fermentation feedstock for fermentation production of a compound, such as ethanol, derived from a herbaceous plant of the family Gramineae or Cucurbitaceae but also as a raw material for sugar production. With the production method of the present embodiment, therefore, both a herbaceous plant-derived compound obtained by fermentation and sugar can be produced from a sugar solution obtained using a herbaceous plant of the family Gramineae or Cucurbitaceae as a raw material.

Furthermore, the production method of the present embodiment, which uses as a fermentation feedstock the reducing sugar-rich sugar solution obtained by the sugar separation step, can yield a higher fermentation efficiency in repeated-batch fermentation than any methods using as a fermentation feedstock a sugar solution obtained without the sugar separation step. This results in an increase in fermentation rate and hence an improvement in the efficiency of production of a compound derived from a herbaceous plant of the family Gramineae or Cucurbitaceae. The reason why the fermentation efficiency can be improved by using as a fermentation feedstock the reducing sugar-rich sugar solution obtained by the sugar separation step has not been clarified; however, the improvement in fermentation efficiency is presumed to be due to the fact that monosaccharides such as glucose and fructose which are easily degradable by the microorganism are concentrated in the reducing sugar-rich sugar solution obtained by the sugar separation step in the present embodiment and the fact that amino acids such as asparagine, arginine, serine, and valine which are favorable for the microorganism are also concentrated in the reducing sugar-rich sugar solution.

### EXAMPLES

Next, the method for producing a compound derived from a herbaceous plant of the family Gramineae or Cucurbitaceae according to the present invention will be specifically described with Examples.

### Example 1

### Preparation of Concentrated Sugar Solution

First, sorghum juice was prepared. In this example, the sorghum juice (sugar solution made from sorghum) was obtained by squeezing sorghum (cultivar: “SIL-05”, acquired from the Togo Field Science and Education Center of Graduate School of Bioagricultural Sciences, Nagoya University) with a juice extractor (manufactured by Okuhara Tekko Co., Ltd.). The obtained sorghum juice (pH: 5.2) contained 62.3 g/L of sucrose, 35.9 g/L of glucose, and 26.8 g/L of fructose.

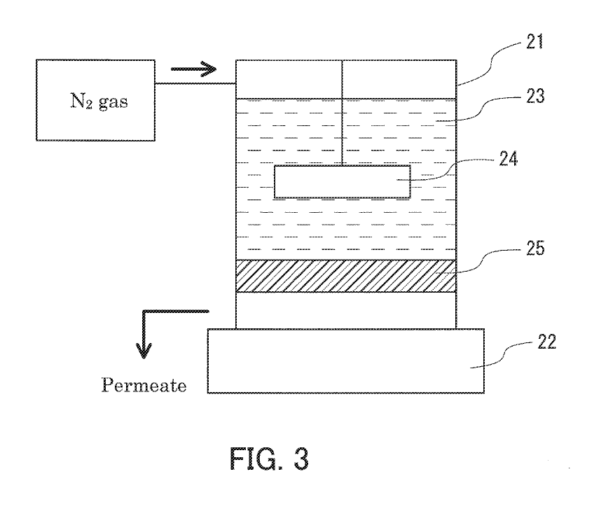

Next, the sorghum juice was filtered through an UF membrane (“RS 50”, manufactured by Nitto Denko Corporation; molecular weight cut off: 150000 Da) to remove residues (UF membrane filtration). The sugar concentration (the concentrations of sucrose, glucose, and fructose) in the sorghum juice resulting from the residue removal was the same as that in the sorghum juice from which the residues had not been removed yet. The UF membrane filtration was carried out with the UF membrane attached to a batch-type flat membrane test cell (“Membrane Master C40-B” manufactured by Nitto Denko Corporation; diameter: 104 mm, height: 147 mm, maximum volume: 380 mL). Specifically, as shown in FIG. 3, a test cell 21 with an UF membrane 25 attached thereto was placed on a magnetic stirrer 22, and sorghum juice 23 contained in the test cell 21 was stirred with a stirring element 24. The filtration was performed at a room temperature of 25° C. while a pressure of 0.5 MPa was applied to the interior of the test cell 21 by nitrogen gas. Residues on the feed side of the UF membrane 25 were removed by this filtration, and the UF membrane permeate was obtained as residue-free sorghum juice.

Next, the residue-free sorghum juice was concentrated using a NF membrane (“ESNA3” manufactured by Nitto Denko Corporation; molecular weight cut off: 150 Da) (NF membrane concentration). The NF membrane concentration was carried out with the batch-type flat membrane test cell (“Membrane Master C40-B” manufactured by Nitto Denko Corporation) used also in the UF membrane filtration. Specifically, as shown in FIG. 3, a test cell 21 with a NF membrane 25 attached thereto was placed on a magnetic stirrer 22, and residue-free sorghum juice 23 contained in the test cell 21 was stirred with a stirring element 24. The NF membrane concentration was performed at a room temperature of 25° C. while a pressure of 2.8 MPa was applied to the interior of the test cell 21 by nitrogen gas. As a result of the NF membrane concentration, a concentrated sugar solution containing sugars concentrated by a factor of about 2.2 was obtained on the feed side of the NF membrane 25. Specifically, the concentrated sugar solution contained 164.4 g/L of sucrose, 60.0 g/L of glucose, and 54.2 g/L of fructose.

### Ethanol Fermentation from Concentrated Sugar Solution

The concentrated sugar solution obtained in the above manner was used for ethanol fermentation. First, a BY4741 strain of Saccharomyces cerevisiae was prepared. This strain was pre-cultured in 5 mL of YPD medium (10 g/L yeast extract, 20 g/L polypeptone, and 20 g/L glucose) at 30° C. and 150 rpm for 24 hours and then further cultured in 500 mL of the same YPD medium for 24 hours. The resulting culture was collected and used as a BY4741 strain. The yeast BY4741 strain was added at a concentration of 50 wet-g/L (corresponding to 10 g/L in a dry state) to the concentrated sugar solution obtained in the manner described above, and ethanol fermentation was performed at 30° C. for 48 hours (first fermentation step). During the fermentation, the solution to be fermented was agitated in a 50-mL bottle (working volume: 10 mL) equipped with an outlet for discharging CO₂ gas. After the 48-hour ethanol fermentation, the resulting fermented solution (first fermented solution) was separated into a solid component and a liquid component by centrifugation, and the liquid component was removed. To the resulting solid component was added a concentrated sugar solution (second concentrated sugar solution) in a volume equal to that of the removed liquid component, and ethanol fermentation was performed at 30° C. for 48 hours (second fermentation step). The third fermentation step, the fourth fermentation step, and the fifth fermentation step were performed in the same manner as the second fermentation step. Namely, repeated-batch fermentation consisting of five fermentation steps was accomplished by using the same yeast and adding only concentrated sugar solutions resulting from NF membrane concentration of a sugar solution obtained using sorghum as a raw material. All of the concentrated sugar solutions used in the first to fifth fermentation steps were the concentrated sugar solution prepared in the manner described above in the present example.

### Measurement of Concentrations of Components in Fermented Solutions

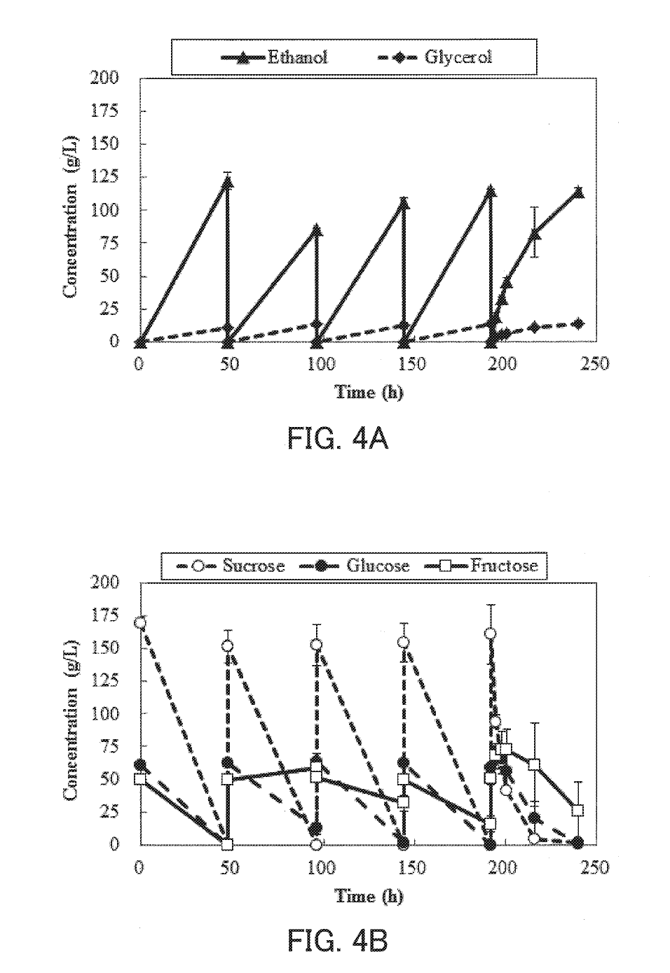

The concentrations of sucrose, glucose, fructose, ethanol, and glycerol in the first to fifth fermented solutions obtained in the first to fifth fermentation steps were measured by using a high-performance liquid chromatograph (manufactured by Shimadzu Corporation) to analyze the liquid components obtained by solid-liquid separation. The results are shown in FIG. 4A and FIG. 4B.

### Example 2

### Preparation of Concentrated Sugar Solution

A concentrated sugar solution was prepared in the same manner as in Example 1.

### Ethanol Fermentation from Concentrated Sugar Solution

In Example 2, unlike in Example 1, YPD medium was not used to culture a BY4741 strain of Saccharomyces cerevisiae, but instead a concentrated sugar solution prepared in the above manner using sorghum as a raw material was used. Namely, in Example 2, yeast was cultured along with fermentation steps. Specifically, ethanol fermentation was performed in the same manner as the ethanol fermentation in Example 1, except that: the concentration of the BY4741 yeast strain added to the concentrated sugar solution in the first fermentation step was 0.04 wet-g/L, which was lower than that in Example 1; the number of repeated fermentation steps was seven; and the volume of the concentrated sugar solution added in each of the repeated fermentation steps was half the volume of the separated liquid component (only half the total volume of the liquid component was replaced by the concentrated sugar solution).

### Measurement of Concentrations of Components in Fermented Solutions

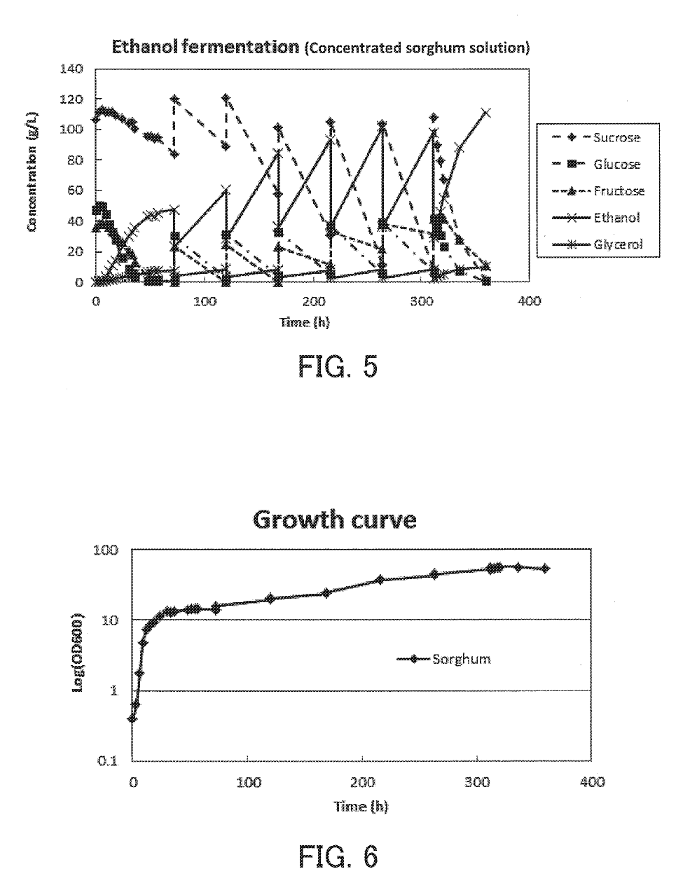

Measurement of the concentrations of components in the fermented solutions was performed in the same manner as in Example 1. The results are shown in FIG. 5.

### Measurement of Yeast Concentration

In Example 2, it was investigated to what extent the yeast was cultured in the concentrated sugar solutions through the first to seventh fermentation steps; namely, the yeast concentration in each fermented solution was determined by turbidimetry. Specifically, the fermented solution was sampled, and the absorbance of the sampled solution at 600 nm was measured with a spectrophotometer (“UVmini-1240” manufactured by Shimadzu Corporation). The results are shown in FIG. 6.

### Comparative Example 1

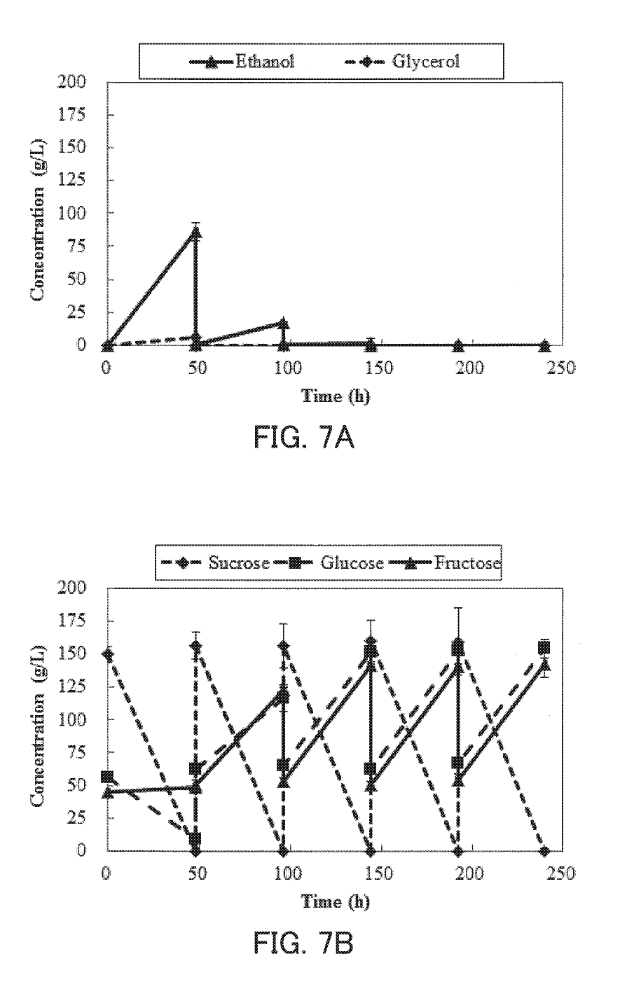

Ethanol fermentation was performed in the same manner as in Example 1, except that a sugar solution containing only sugars (only sucrose, glucose, and fructose; specifically, 149 g/L of sucrose, 56 g/L of glucose, and 46 g/L of fructose) was used for the ethanol fermentation instead of the concentrated sugar solution which was prepared using sorghum as a raw material and which was used in Example 1. Measurement of the concentrations of components in fermented solutions was performed in the same manner as in Example 1. The results are shown in FIG. 7A and FIG. 7B.

### Comparative Example 2

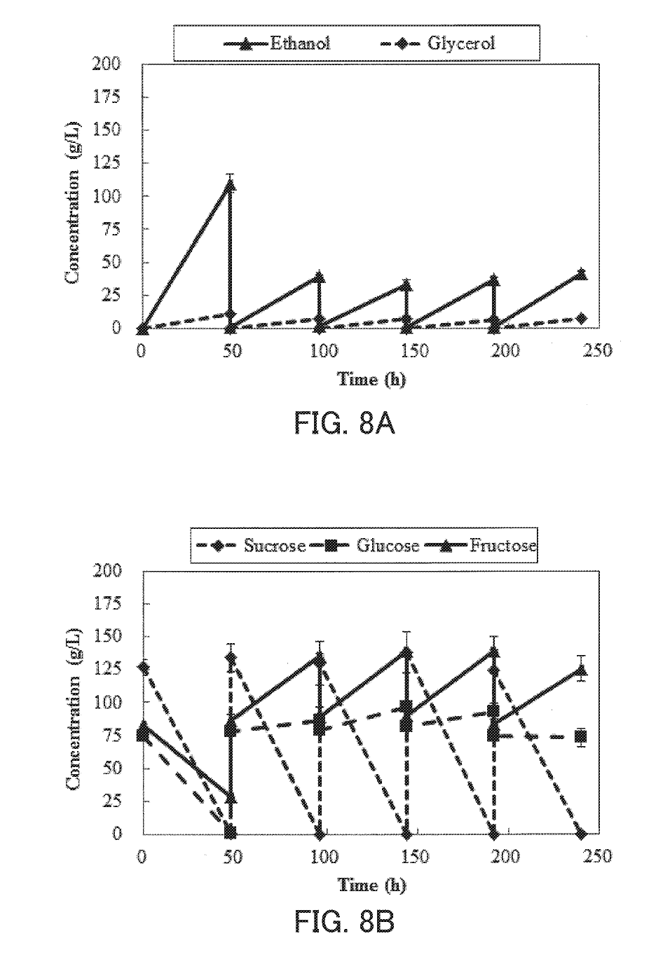

Ethanol fermentation was performed in the same manner as in Example 1, except that a sugar solution containing only molasses (127 g/L of sucrose, 75 g/L of glucose, and 83 g/L of fructose) was used for the ethanol fermentation instead of the concentrated sugar solution which was prepared using sorghum as a raw material and which was used in Example 1. Measurement of the concentrations of components in fermented solutions was performed in the same manner as in Example 1. The results are shown in FIG. 8A and FIG. 8b.

In the repeated-batch fermentation of Example 1, as seen from FIG. 4A, the yeast continued to produce a high concentration of ethanol throughout the five steps of the repeated-batch fermentation, despite the use of only concentrated sugar solutions supplemented with no nitrogen source. For example, the concentration of ethanol obtained by the fifth ethanol fermentation was 113.7±3.1 g/L, which was comparable to the concentration (115.2±7.3 g/L) of ethanol obtained by the first ethanol fermentation.

Further, the result shown in FIG. 6 demonstrated that yeast can be cultured by a concentrated sugar solution obtained using sorghum as a raw material even without supplementation with a nutrient substance such as a nitrogen source. In Example 2, the concentration of ethanol produced by the first and second ethanol fermentation was low, and this can be attributed to a low yeast concentration due to insufficient growth of the yeast. In the third and subsequent ethanol fermentation in which the yeast was sufficiently grown, as shown in FIG. 5, the yeast produced a high concentration of ethanol with the use of only concentrated sugar solutions. As seen from the foregoing, in Example 2 employing yeast cultured in a concentrated sugar solution obtained using sorghum as a raw material, the yeast continued to produce a high concentration of ethanol throughout several steps of the repeated-batch fermentation as in Example 1, despite the use of only concentrated sugar solutions supplemented with no nitrogen source.

In Comparative Example 1 where repeated-batch fermentation was performed using, instead of a concentrated sugar solution obtained using sorghum as a raw material, a sugar solution containing only sugars without supplementation with any nitrogen source, the concentration of produced ethanol in the second fermentation was very low, and no ethanol was produced in the third and subsequent fermentation, as seen from FIG. 7A. In Comparative Example 2 where repeated-batch fermentation was performed using a sugar solution containing molasses without supplementation with any nitrogen source, as seen from FIG. 8A, the concentration of produced ethanol in the second and subsequent ethanol fermentation was much lower than the concentration of produced ethanol in the first ethanol fermentation.

The above results demonstrated that when a concentrated sugar solution obtained by using a NF membrane to concentrate a sugar solution obtained using sorghum as a raw material is used as a fermentation feedstock for fermentation steps in repeated-batch fermentation, yeast can maintain its fermentation capacity and continue to produce a high concentration of ethanol without supplementation with any nitrogen source.

### Example 3

### Sugar Separation Step

First, sorghum juice was prepared. In this example, the sorghum juice (sugar solution made from sorghum) was obtained by squeezing sorghum (cultivar: “SIL-05”, acquired from the Togo Field Science and Education Center of Graduate School of Bioagricultural Sciences, Nagoya University) with a juice extractor (manufactured by Okuhara Tekko Co., Ltd.). The obtained sorghum juice (pH: 5.2) contained 84.0 g/L of sucrose, 26.2 g/L of glucose, and 18.0 g/L of fructose.

Next, the sorghum juice was filtered through an UF membrane (“RS 50”, manufactured by Nitto Denko Corporation; molecular weight cut off: 150000 Da) to remove residues (UF membrane filtration). The sugar concentration (the concentrations of sucrose, glucose, and fructose) in the sorghum juice resulting from the residue removal was the same as that in the sorghum juice from which the residues had not been removed yet. The UF membrane filtration was carried out with the UF membrane attached to a batch-type flat membrane test cell (“Membrane Master C40-B” manufactured by Nitto Denko Corporation; diameter: 104 mm, height: 147 mm, maximum volume: 380 mL). Specifically, as shown in FIG. 3, a test cell 21 with an UF membrane 25 attached thereto was placed on a magnetic stirrer 22, and sorghum juice 23 contained in the test cell 21 was stirred with a stirring element 24. The filtration was performed at a room temperature of 25° C. while a pressure of 0.5 MPa was applied to the interior of the test cell 21 by nitrogen gas. Residues on the feed side of the UF membrane 25 were removed by this filtration, and the UF membrane permeate was obtained as residue-free sorghum juice.

Next, a NF membrane (“NTR-7450” manufactured by Nitto Denko Corporation) was used to separate the residue-free sorghum juice into a sucrose-rich sugar solution and a reducing sugar-rich sugar solution (sugar separation). The NF membrane used for the sugar separation, “NTR-7450”, is a membrane for which the value of sucrose rejection performance/glucose rejection performance is 2.4 and which has a molecular weight cut off of about 1000 Da. The sugar separation was carried out with the batch-type flat membrane test cell (“Membrane Master C40-B” manufactured by Nitto Denko Corporation) used also in the UF membrane filtration. Specifically, as shown in FIG. 3, a test cell 21 with a NF membrane 25 attached thereto was placed on a magnetic stirrer 22, and residue-free sorghum juice 23 contained in the test cell 21 was stirred with a stirring element 24. The sugar separation was performed at a room temperature of 25° C. while a pressure of 2.0 MPa was applied to the interior of the test cell 21 by nitrogen gas. As a result of the sugar separation, a sugar solution (sucrose-rich sugar solution) containing an increased concentration of sucrose and a decreased concentration of reducing sugars such as glucose and fructose was obtained on the feed side of the NF membrane 25. This sucrose-rich sugar solution was five-fold diluted and further subjected to sugar separation (second sugar separation) using a NF membrane (“NTR-7450” manufactured by Nitto Denko Corporation). The second sugar separation was performed in the same manner as the first sugar separation to give a sugar solution containing an increased concentration of sucrose and a decreased concentration of reducing sugars such as glucose and fructose on the feed side of the NF membrane 25. The sucrose-rich sugar solution finally obtained was used as a raw material for the sugar production step. The finally-obtained sucrose-rich sugar solution contained 143.2 g/L of sucrose, 8.5 g/L of glucose, and 4.5 g/L of fructose. The sugar solution that passed through the NF membrane 25 in the first and second sugar separation was used as a reducing sugar-rich sugar solution in the subsequent step of preparing a concentrated sugar solution.

### Preparation of Concentrated Sugar Solution

A concentrated sugar solution was prepared in the same manner as in Example 1, except that the reducing sugar-rich sugar solution obtained in the sugar separation step was used as a raw material for sugar concentration. The resulting concentrated sugar solution contained 96.3 g/L of sucrose, 76.1 g/L of glucose, and 55.5 g/L of fructose.

### Ethanol Fermentation from Concentrated Sugar Solution

The concentrated sugar solution obtained in the above manner was used for ethanol fermentation. First, a BY4741 strain of Saccharomyces cerevisiae was prepared. This strain was pre-cultured in 5 mL of YPD medium (10 g/L yeast extract, 20 g/L polypeptone, and 20 g/L glucose) at 30° C. and 150 rpm for 24 hours and then further cultured in 500 mL of the same YPD medium for 48 hours. The resulting culture was collected and used as a BY4741 strain. The yeast BY4741 strain was added at a concentration of 50 wet-g/L (corresponding to 10 g/L in a dry state) to the concentrated sugar solution obtained in the manner described above, and ethanol fermentation was performed at 30° C. for 48 hours (first fermentation step). During the fermentation, the solution to be fermented was agitated in a 50-mL bottle (working volume: 10 mL) equipped with an outlet for discharging CO₂ gas. After the 24-hour ethanol fermentation, the resulting fermented solution (first fermented solution) was separated into a solid component and a liquid component by centrifugation, and the liquid component was removed. To the resulting solid component was added a concentrated sugar solution (second concentrated sugar solution) in a volume equal to that of the removed liquid component, and ethanol fermentation was performed at 30° C. for 24 hours (second fermentation step). The third fermentation step, the fourth fermentation step, and the fifth fermentation step were performed in the same manner as the second fermentation step. Namely, repeated-batch fermentation consisting of five fermentation steps was accomplished by using the same yeast and adding only concentrated sugar solutions resulting from NF membrane concentration of a reducing sugar-rich sugar solution obtained through sugar separation of a sugar solution obtained using sorghum as a raw material. All of the concentrated sugar solutions used in the first to fifth fermentation steps were the concentrated sugar solution prepared in the manner described above in the present example.

### Measurement of Concentrations of Components in Fermented Solutions

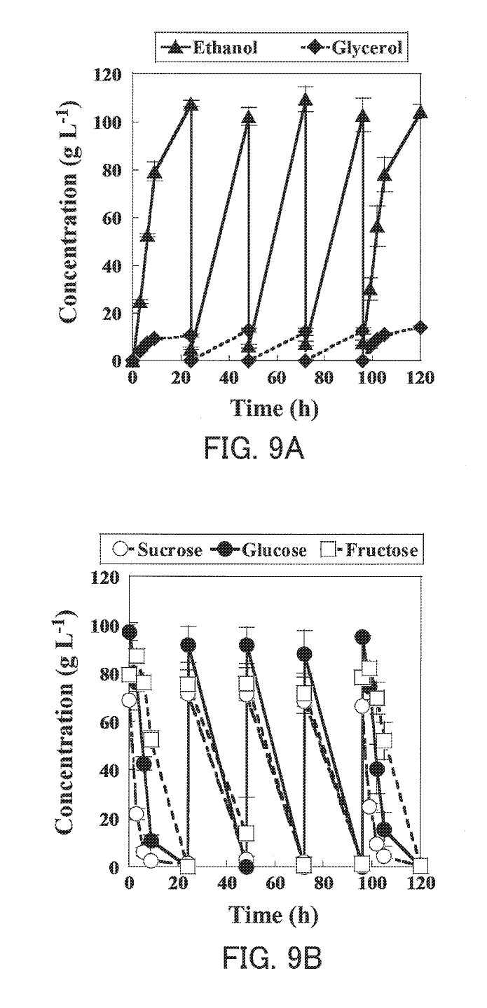

The concentrations of sucrose, glucose, fructose, ethanol, and glycerol in the first to fifth fermented solutions obtained in the first to fifth fermentation steps were measured by using a high-performance liquid chromatograph (manufactured by Shimadzu Corporation) to analyze the liquid components obtained by solid-liquid separation. The results are shown in FIG. 9A and FIG. 9B.

As seen by comparing the result for Example 1 (FIG. 4A) and the result for Example 3 (FIG. 9A), the fermentation steps of Example 3, in which the sugar separation step was performed, gave a high concentration of ethanol comparable to that produced by the fermentation steps of Example 1, despite the fact that the time of the fermentation steps of Example 3 was 24 hours corresponding to half the time (48 hours) of the fermentation steps of Example 1. This result reveals that when a reducing sugar-rich sugar solution obtained by the sugar separation step is used as a fermentation feedstock for repeated-batch fermentation, the fermentation efficiency and hence the fermentation rate are increased as compared to when a sugar solution prepared without the sugar separation step is used as a fermentation feedstock.

### INDUSTRIAL APPLICABILITY

With the production method of the present invention, a compound derived from a herbaceous plant of the family Gramineae or Cucurbitaceae, such as ethanol, can be produced by repeated-batch fermentation efficiently and simply. The present invention can therefore be used in a wide variety of applications where a sugar solution obtained using a herbaceous plant of the family Gramineae or Cucurbitaceae as a raw material is converted to a chemical product by fermentation.

1. A method for producing a compound derived from a herbaceous plant of the family Gramineae or Cucurbitaceae, comprising:
adding at least one microorganism selected from yeast, Escherichia coli, and bacteria of the genus Corynebacterium to a first concentrated sugar solution to ferment the first concentrated sugar solution and subjecting the resulting first fermented solution to solid-liquid separation to separate the first fermented solution into a solid component and a liquid component, the first concentrated sugar solution being obtained by using at least one of a nanofiltration membrane and a reverse osmosis membrane to concentrate a sugar solution obtained using a herbaceous plant of the family Gramineae or Cucurbitaceae as a raw material; and
adding a second concentrated sugar solution to the solid component obtained from the first fermented solution to ferment the second concentrated sugar solution with the microorganism used to ferment the first concentrated sugar solution, and subjecting the resulting second fermented solution to solid-liquid separation to separate the second fermented solution into a solid component and a liquid component, the second concentrated sugar solution being obtained by using at least one of a nanofiltration membrane and a reverse osmosis membrane to concentrate a sugar solution obtained using a herbaceous plant of the family Gramineae or Cucurbitaceae as a raw material,
wherein the compound derived from the herbaceous plant of the family Gramineae or Cucurbitaceae is obtained from the liquid component obtained from at least one of the first fermented solution and the second fermented solution.

2. The method according to claim 1, wherein no nitrogen source is added to the solid component used to ferment the second concentrated sugar solution.

3. The method according to claim 2, wherein only the second concentrated sugar solution is added to the solid component used to ferment the second concentrated sugar solution.

4. The method according to claim 1, further comprising preparing a concentrated sugar solution by using at least one of a nanofiltration membrane and a reverse osmosis membrane to concentrate a sugar solution obtained using a herbaceous plant of the family Gramineae or Cucurbitaceae as a raw material,
wherein the first concentrated sugar solution and the second concentrated sugar solution are each a portion of the concentrated sugar solution obtained.

5. The method according to claim 4, further comprising using a nanofiltration membrane to separate a sugar solution obtained using a herbaceous plant of the family Gramineae or Cucurbitaceae as a raw material into a sucrose-rich sugar solution and a reducing sugar-rich sugar solution,
wherein the reducing sugar-rich sugar solution is used as the sugar solution to prepare the concentrated sugar solution.

6. The method according to claim 5, further comprising producing sugar using the sucrose-rich sugar solution.

7. A method for producing a compound derived from a herbaceous plant of the family Gramineae or Cucurbitaceae, comprising:
a first fermentation comprising adding at least one microorganism selected from yeast, Escherichia coli, and bacteria of the genus Corynebacterium to a first concentrated sugar solution to ferment the first concentrated sugar solution and subjecting the resulting first fermented solution to solid-liquid separation to separate the first fermented solution into a solid component and a liquid component, the first concentrated sugar solution being obtained by using at least one of a nanofiltration membrane and a reverse osmosis membrane to concentrate a sugar solution obtained using a herbaceous plant of the family Gramineae or Cucurbitaceae as a raw material;
a second fermentation comprising adding a second concentrated sugar solution to the solid component obtained from the first fermented solution to ferment the second concentrated sugar solution with the microorganism used to ferment the first concentrated sugar solution and subjecting the resulting second fermented solution to solid-liquid separation to separate the second fermented solution into a solid component and a liquid component, the second concentrated sugar solution being obtained by using at least one of a nanofiltration membrane and a reverse osmosis membrane to concentrate a sugar solution obtained using a herbaceous plant of the family Gramineae or Cucurbitaceae as a raw material; and
third to N-th fermentations performed after the second fermentation, where N is an integer of 3 or more,
wherein in an L-th fermentation included in the second to N-th fermentations, where L is an integer satisfying 2≤L≤N, an L-th concentrated sugar solution obtained by using at least one of a nanofiltration membrane and a reverse osmosis membrane to concentrate a sugar solution obtained using a herbaceous plant of the family Gramineae or Cucurbitaceae as a raw material is added to a solid component obtained by solid-liquid separation of a fermented solution obtained in a fermentation selected from the first to (L−1)-th fermentations to ferment the L-th concentrated sugar solution with the microorganism used in the fermentation selected from the first to (L−1)-th fermentations, and the resulting L-th fermented solution is subjected to solid-liquid separation to separate the L-th fermented solution into a solid component and a liquid component, and
wherein a compound derived from a herbaceous plant of the family Gramineae or Cucurbitaceae is obtained from the liquid component obtained in at least one fermentation selected from the first to N-th fermentations.

8. The method according to claim 7, wherein in the L-th fermentation, the L-th concentrated sugar solution is added to a solid component obtained in the (L−1)-th fermentation to ferment the L-th concentrated sugar solution with the microorganism used in the (L−1)-th fermentation.

9. The method according to claim 7, wherein in the L-th fermentation, no nitrogen source is added to the solid component.

10. The method according to claim 9, wherein in the L-th fermentation, only the L-th concentrated sugar solution is added to the solid component.

11. The method according to claim 7, further comprising, prior to the first fermentation, preparing a concentrated sugar solution by using at least one of a nanofiltration membrane and a reverse osmosis membrane to concentrate a sugar solution obtained using a herbaceous plant of the family Gramineae or Cucurbitaceae as a raw material,
wherein the first concentrated sugar solution and the L-th concentrated sugar solution are each a portion of the concentrated sugar solution.

12. The method according to claim 11, further comprising, prior to preparing the concentrated sugar solution, using a nanofiltration membrane to separate a sugar solution obtained using a herbaceous plant of the family Gramineae or Cucurbitaceae as a raw material into a sucrose-rich sugar solution and a reducing sugar-rich sugar solution,
wherein the reducing sugar-rich sugar solution is used as the sugar solution to prepare the concentrated sugar solution.

13. The method according to claim 12, further comprising producing sugar using the sucrose-rich sugar solution.

14. The method according to claim 1, further comprising culturing the microorganism with a concentrated sugar solution obtained by using at least one of a nanofiltration membrane and a reverse osmosis membrane to concentrate a sugar solution obtained using a herbaceous plant of the family Gramineae or Cucurbitaceae as a raw material.

15. The method according to claim 1, wherein the sugar solution obtained using a herbaceous plant of the family Gramineae or Cucurbitaceae as a raw material is subjected to a pretreatment using an ultrafiltration membrane before the sugar solution is concentrated using at least one of the nanofiltration membrane and the reverse osmosis membrane.

16. The method according to claim 1, wherein the compound derived from a herbaceous plant of the family Gramineae or Cucurbitaceae is ethanol.

17. The method according to claim 1, wherein the herbaceous plant is sorghum.
